(19) United States
(12) Patent Application Publication
(10) Pub. No.: US 2009/0275862 A1
(43) Pub. Date: Nov. 5, 2009

(54) GUIDEWIRE AND METHOD OF MAKING SAME

(75) Inventor: James C. Elsesser, Bloomington, IN (US)

Correspondence Address: BRINKS HOFER GILSON & LIONE/CHICAGO/COOK, PO BOX 10395, CHICAGO, IL, 60610, US

(73) Assignee: COOK INCORPORATED, BLOOMINGTON, IN (US)

(21) Appl. No.: 12/112,419

(22) Filed: Apr. 30, 2008

(51) Int. Cl. A61M 25/09

(52) U.S. Cl. 600/585; 29/428; 29/469.5

(57) ABSTRACT

In at least one embodiment of the present invention, a method for making a guidewire is provided. The method comprises removing material from a distal portion of a core wire to define a distal tip extending from a proximal portion of the core wire. A collar is positioned adjacent to the distal tip about the proximal portion.

### BACKGROUND OF THE INVENTION

1. Field of the Invention

The present invention relates to medical devices. More particularly, the invention relates to guidewires and a method for making guidewires.

2. Background

Often elongated, flexible guidewires are used to gain access to specific inner areas of the body. The guidewires may enter the body through a small opening and travel to parts of the body through body channels. For example, guidewires may be passed through the body via peripheral blood vessels, gastrointestinal tract, or the urinary tract. Guidewires are commercially available and are currently used in cardiology, gastroenterology, urology, and radiology. Once in place at a desired location in the body, guidewires are commonly used as guides for the introduction of additional medical instruments, e.g. catheters.

To assist in advancing a guidewire through a predetermined body vessel, such as an artery or other channel, the guidewire typically includes a generally flexible body portion, which is resistant to kinking, and a forward end portion of increased flexibility. The end portion or distal tip portion often terminates in a smoothly rounded tip. The body portion may include a core wire of stainless steel or other metal. The core wire is appropriately dimensioned in cross-section to provide a controlled degree of flexibility to the body portion. In order to provide greater flexibility to the guidewire at its distal end, the forward end of the core wire may include a section of lesser diameter and hence, of greater flexibility. Typically, around this section of lesser diameter a flexible helically wound wire or spring coil is attached via soldering or welding, forming a solder or weld ball at the distal extreme of the guidewire. The ball is then ground to form the smoothly rounded tip made from solder or weld material.

One concern, however, is that the solder or weld balls may not be consistently formed relative to each other when numerous guidewires are produced. Moreover, with the increasing number of different medical procedures, various tip configurations may be also desirable. Some of these tip configurations may have shapes (e.g. narrow and/or more intricate cross-sections) which require more exact machining and/or grinding of the ball. If the ball is not consistently formed, grinding material away from the ball to form the tip may locally thin-out and/or weaken the attachment of the tip to the core wire which may also facilitate the tip or pieces of the tip becoming detached from the core wire during the medical procedure. If any of these pieces become detached within the body vessel of the patient, they may become problematic for the patient.

### BRIEF SUMMARY OF THE INVENTION

In at least one embodiment of the present invention, a method for making a guidewire is provided. The method comprises removing material from a distal portion of a core wire to define a distal tip. The distal tip extends from a proximal portion of the core wire. The distal tip has a maximum cross-sectional dimension not exceeding a maximum diameter of the proximal portion. A collar having a lumen is positioned adjacent to the distal tip about the proximal portion. Positioning the collar includes advancing the distal tip through the lumen.

In one aspect, the collar is formed exclusively from the core wire.

In another aspect, material is removed from a distal section of the proximal portion to define a recessed portion. The collar is placed about the recessed portion.

Further objects, features, and advantages of the present invention will become apparent from consideration of the following description and the appended claims when taken in connection with the accompanying drawings.

### BRIEF DESCRIPTION OF THE DRAWINGS

FIG. 1 is a flow chart for a method of making a guidewire in accordance with one example of the present invention;

FIG. 2 is a side view of a core wire in accordance with an embodiment of the present invention;

FIG. 3a is a side view of a guidewire in accordance with an embodiment of the present invention;

FIG. 3b is a side view of a portion of a guidewire in accordance with another embodiment of the present invention;

FIG. 3c is a side view of a portion of a guidewire in accordance with yet another embodiment of the present invention;

FIG. 4a is a side view of a guidewire in accordance with an embodiment of the present invention;

FIG. 4b is a cross sectional view of the guidewire depicted in FIG. 4a;

FIG. 4c is a cross-sectional view of a guidewire in accordance with an embodiment of the present invention;

FIG. 5 is a cross-sectional view of a guidewire in accordance with another embodiment of the present invention;

FIG. 6a is an exploded view of a catheter kit for a body vessel in accordance with one embodiment of the present invention;

FIG. 6b is a side view of the catheter kit in accordance with another embodiment of the present invention; and

FIG. 7 is a cross-sectional environmental view of a guidewire deployed in a body vessel.

### DETAILED DESCRIPTION OF THE INVENTION

Detailed embodiments of the present invention are disclosed herein. It is understood however, that the disclosed embodiments are merely exemplary of the invention and may be embodied in various and alternative forms. The figures are not necessarily to scale; some figures may be configured to show the details of a particular component. Therefore, specific structural and functional details disclosed herein are not to be interpreted as limiting but merely as a representative basis with the claims and for teaching one skilled in the art to practice the present invention.

Examples of the present invention seek to overcome some of the concerns associated with providing a guidewire for guiding various medical devices through a body vessel or cavity of a patient while minimizing the possibility of the distal tip of the guidewire becoming detached during the medical procedure.

Employing the principles of the present invention is, for example, a guidewire, a method for making the guidewire, and a catheter kit. The guidewire, which is made from the method and is utilized in the kit, has an elongated core wire with a forward end that forms the distal tip of the guide wire. The distal tip is integrally connected to the core wire and is preferably formed exclusively from the core wire. In one example, numerous core wires may be produced (e.g. by pultrusion, extrusion or stamping with precision dies) which are dimensionally consistent relative to each other. Machining or grinding of dimensionally consistent core wires allows for more precise material deletion to form corresponding tips for the core wires. Thus, various tip configurations may be produced without locally thinning-out and/or weakening the attachment of the tip to the core wire, which preferably minimizes the possibility of the tip or pieces of the tip becoming detached from the core wire while being deployed within a body vessel of the patient.

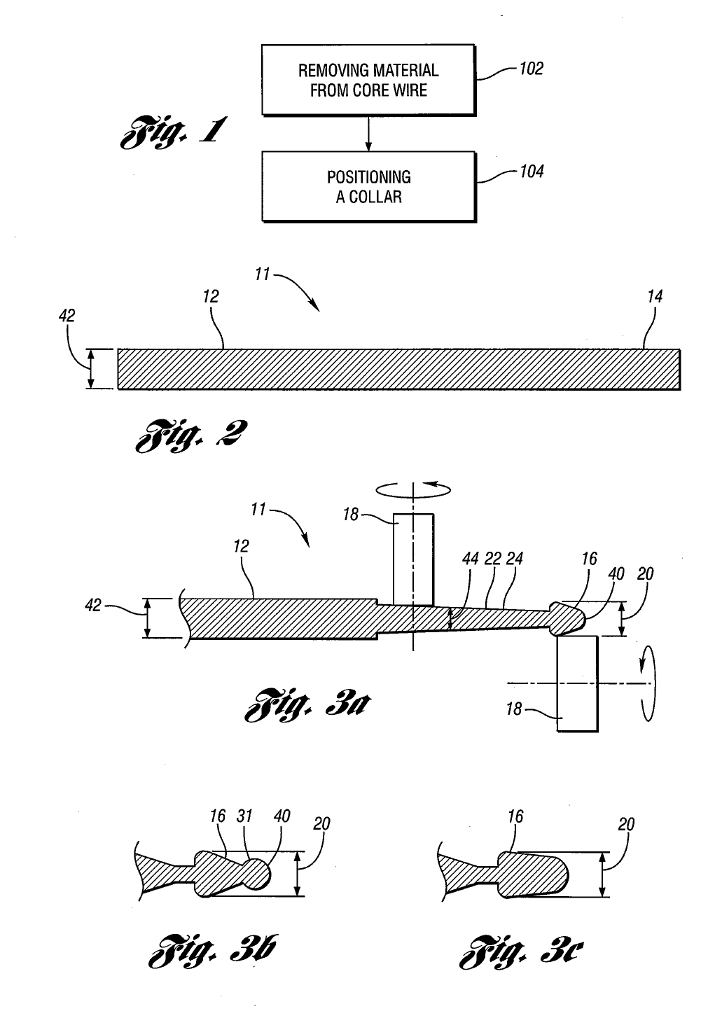

Referring to FIGS. 1-3c, a method for making a guidewire is provided. As illustrated in FIG. 2, an elongated core wire 11 is provided. The core wire 11 has a proximal portion 12 and a distal portion 14 that extends from the proximal portion 12. The core wire 11 may be made of stainless steel or any other suitable material known to those skilled in the art.

Material is removed 102 from the distal portion 14 of the core wire 11 to form a distal tip 16. In at least one embodiment, the distal tip 16 is monolithically formed from the core wire 11. For example, the distal tip 16 may be integrally connected with and formed exclusively from the core wire 11.

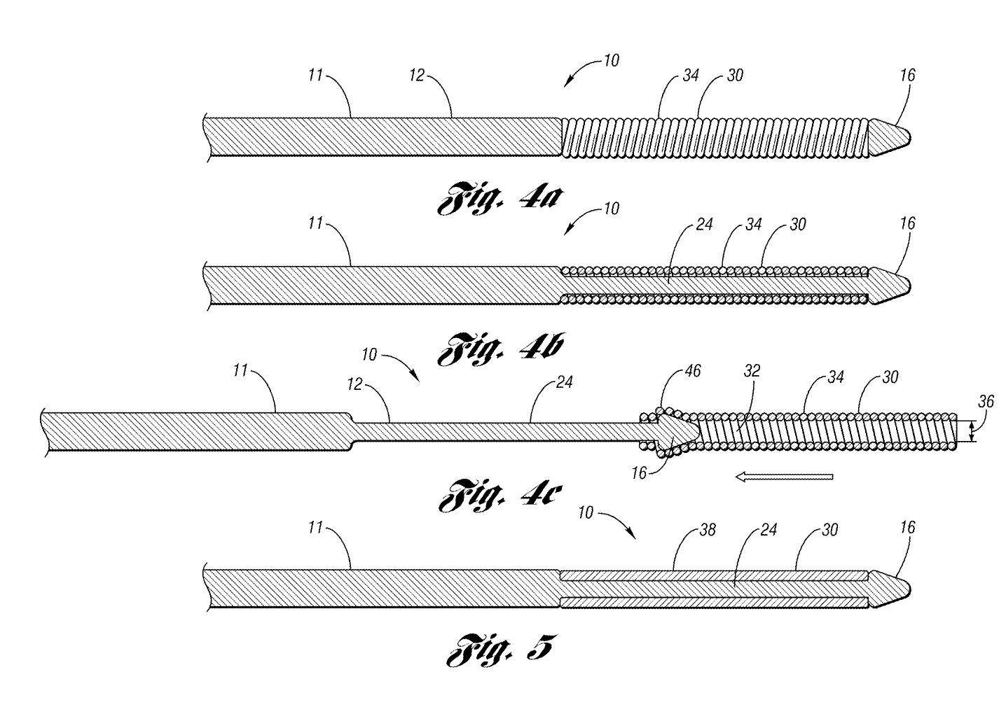

As illustrated in FIGS. 3a-3c, the distal tip 16 may have different shapes with varying dimensions and geometries. The distal tip 16 may have a variable outside dimension 20 (e.g. axial cross-sectional dimension) that is configured to taper distally along the length of the tip 16 (FIG. 3a and 3b). Alternatively, the variable outside dimension 20 may be configured to flare distally along the length of the distal tip (FIG. 3b at transition 31). In one example, the tip 16 has a rounded portion 40 at its distal extreme. In another example, the maximum outside dimension 20 of the distal tip 16 does not exceed a maximum diameter 42 of the proximal portion 12. Although the core wire 11 may have various outside diameters, an outside diameter 42 not exceeding about 0.020 inches may be preferred for many medical procedures.

As depicted in FIG. 3a, material may be removed from the distal portion 14 by machining or grinding the core wire 11 with a grinding wheel 18 or mill. Because the tip 16 may have a shape with varying dimensions and geometries, the grinding wheel 18 may need to be accurately controlled for movement over numerous axes. This may be accomplished by using an automated computer numerically controlled (CNC) multi-axis grinding machine. Preferably, the CNC grinding machine is capable of controlled movement over at least the X and Y axis. One such machine is a CAM.2 Profile Grinder manufactured by Glebar Company Incorporated. The CAM.2 Profile Grinder has direct interface with CAD/CAM and includes a fully integrated multi-axis servo controller. This arrangement may allow for machining of very intricate shapes which have been designed using a CAD based program. In one example, the grinding machine is capable of machining the shape of the distal tip 16 to within 0.1 microns of the targeted dimensions.

Material may also be removed from the core wire 11 at a distal section 22 of the proximal portion 12 to form a recessed portion 24. The recessed portion 24 has a reduced axial cross-sectional dimension 44 relative to the maximum or base diameter 42 of core wire 11. This reduced dimension 44 may provide the forward end of the core wire 11 with greater flexibility.

Referring to FIGS. 1-5, in at least one embodiment the reduced axial cross-sectional dimension 44 is less than the maximum outside dimension 20 of the tip 16. A collar 30 may be positioned 104 adjacent to the distal tip 16 about the proximal portion 12 and more preferably, about the recessed portion 24. In one example, the greater outside dimension 20 of the tip facilitates retaining the collar 30 about the recessed portion 24.

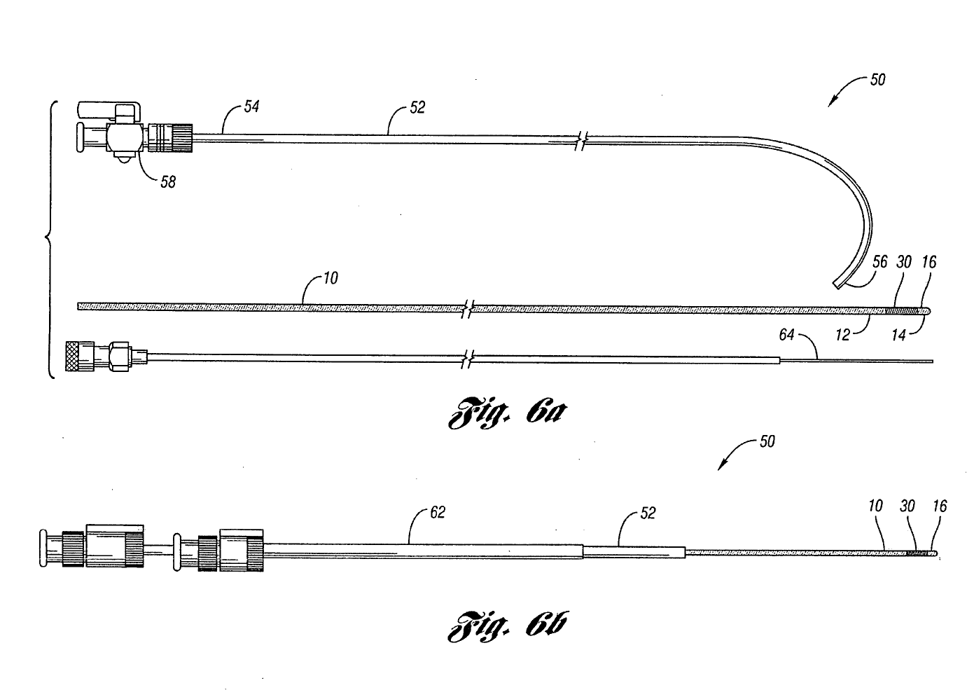

The collar 30 may be positioned about the proximal portion 12 by advancing the distal tip 16 through a lumen 32 of the collar 30. In one example and as illustrated in FIGS. 4a-4c, the collar 30 may be a coil spring 34 which has a lumen 32 with a diameter 36 which is smaller than the maximum outside dimension 20 of the tip 16. The coil spring 34 may elastically deform 46 about the distal tip 16 while being advanced over the tip 16, permitting the coil spring 34 to be positioned on proximal portion 12 of the core wire 11. Once advanced over the tip 16, the coil spring 34 elastically returns to fit about the recessed portion 24 with the tip 16 preferably retaining the coil spring 34 thereon.

In another example, the collar 30 is shrink tubing 38 which may also be advanced over the tip 16 to be positioned about the proximal portion 12. The shrink tubing 38 may be shrunk or reduced in diameter to fit preferably about the recessed portion 24 and retained thereon by the tip and/or frictional engagement with the core wire 11. The shrink tubing 38 may be shrunk, for example, by applying heat or ultraviolet radiation to facilitate crystallization and/or cross-linking of the tubing material. Compositions and processing for shrink tubings 38 are generally known in the art and any suitable shrink tubing and associated processing to shrink the tubing 38 may be used.

In at least one embodiment, the collar 30 is attached to the core wire 11 to form the guide wire 10. The collar 30 may be bonded to the proximal portion 12 with adhesive. Alternatively, the collar 30 may be attached to the proximal portion with solder or by welding. Other suitable means known to those skilled in the art for attaching a collar 30 to the core wire 11 may also be used. Preferably, attachment of the collar 30 to the core wire 11 does not leave remnants of adhesive, solder or welding material on the tip 16.

The collar 30 may also include a radio pacifier that is detectable by X-ray and/or fluoroscopic visualization. The radio pacifier may be incorporated directly into the material of the collar 30 or coated thereon. Alternatively, the radiopacifier may be included as part of the adhesive, welding material or solder that is used to attach the collar 30 to the core wire 11.

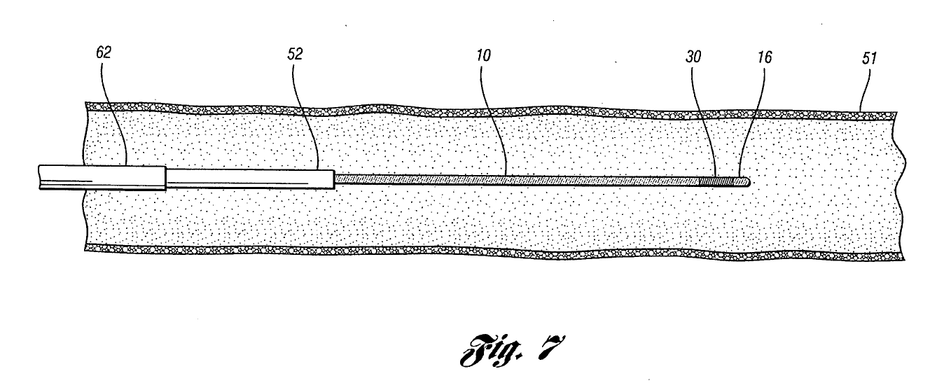

Referring to FIGS. 6a-7, a catheter kit 50 for a body vessel 51 is provided. As shown, the kit 50 includes a microcatheter 52 preferably made from a soft, flexible material such as silicone or any other suitable material. Generally, the microcatheter 52 has a proximal end 54, a distal end 56, and a plastic adapter or hub 58 to receive a medical device (not shown), e.g., angioplasty balloon, stent, occluding device, etc., to be advanced therethrough. In this embodiment, the inside diameter of the microcatheter 52 may range between 0.014 and 0.027 inch.

The kit 50 further includes the guide wire 10 as discussed in the foregoing paragraphs. The guide wire 10 provides a guide catheter 62 (discussed in more detail below) a path during insertion of the guide catheter 62 within the body vessel 51. The size of the wire guide 10 is based on the inside diameter of the guide catheter 62.

The guide catheter 62 or sheath is typically made from polytetrafluoroethylene (PTFE) and is for percutaneously introducing the microcatheter 52 into a body vessel 51. Of course, any other suitable material may be used without falling beyond the scope or spirit of the present invention. The guide catheter 62 may have a size of between about 4-French to 8-French and allows the microcatheter 52 to be inserted therethrough to a desired location in the body vessel 51. The guide catheter 62 receives the microcatheter 52 and provides stability of the microcatheter 52 at a desired location within the body vessel 51. For example, the guide catheter 52 may stay stationary within a common visceral artery, e.g., a common hepatic artery, and adds stability to the microcatheter 52 as the microcatheter 52 is advanced through the guide catheter 62 to a desired point in a connecting artery, e.g., the left or right hepatic artery.

When the distal end 56 of the microcatheter 52 is at the desired point in the body vessel 51, the medical device may be loaded at the proximal end 54 of the microcatheter 52 and is advanced through the microcatheter 52 for deployment through the distal end 56. In one embodiment, a pushwire 64 is used to mechanically advance or push the medical device through the microcatheter 52. The size of the pushwire 64 used depends on the diameter of the microcatheter 52.

It is to be understood that the catheter kit 50 described above is merely one example of a kit that may be used with the guidewire 10, which is for being passed through a body vessel 51 or cavity. Of course, other kits, assemblies, and systems may be used with the guidewire 10 without falling beyond the scope or spirit of the present invention.

As a person skilled in the art will readily appreciate, the above description is meant as an illustration of the implementation of the principles of this invention. This description is not intended to limit the scope of application of this invention in that the invention is susceptible to modification, variation, and change, without departing from the spirit of this invention, as defined in the following claims.

1. A method for making a guidewire comprising:
removing material from a distal portion of a core wire to define a distal tip extending from a proximal portion of the core wire, the distal tip having a maximum cross-sectional dimension not exceeding a maximum diameter of the proximal portion;
removing material from a distal section of the proximal portion of the core wire to define a recessed portion; and
positioning a collar adjacent to the distal tip about the proximal portion including advancing the distal tip through a lumen of the collar and placing the collar about the recessed portion.

2. The method according to claim 1 wherein the distal tip is formed exclusively from the core wire.

3. The method according to claim 1 wherein the maximum cross-sectional dimension of the distal tip exceeds a diameter of the lumen of the collar.

4. The method according to claim 3 wherein the collar elastically deforms during the step of advancing the distal tip to permit positioning of the collar about the proximal portion of the core wire.

5. The method according to claim 1 wherein the collar is a coil spring.

6. The method according to claim 1 wherein the collar is a shrink tubing and the step of positioning includes shrinking the shrink tubing about the proximal portion.

7. The method according to claim 1 wherein the step of removing includes machining the distal portion of the core wire with a multi-axis grinding machine to form the distal tip.

8. The method according to claim 1 wherein the recessed portion retains the collar thereon.

9. The method according to claim 1 wherein the step of positioning includes attaching the collar to the proximal portion by one of adhesive bonding, soldering and welding.

10. The method according to claim 1 wherein the distal tip has a variable outside dimension configured to at least one of taper and flare distally along a length of the distal tip.

11. The method according to claim 1 wherein the maximum diameter of the proximal portion does not exceed about 0.020 inches.

12. A method for making a guidewire comprising:
providing a core wire having a proximal portion and a distal portion extending therefrom;
removing material from the distal portion to define a distal tip; and
positioning a collar adjacent to the distal tip about the proximal portion.

13. The method according to claim 1 wherein the step of positioning includes advancing the distal tip through a lumen of the collar.

14. The method according to claim 13 wherein the distal tip has a maximum cross-sectional dimension exceeding a diameter of the lumen of the collar, and wherein the collar elastically deforms during the step of advancing to permit positioning of the collar about the proximal portion of the core wire.

15. The method according to claim 12 wherein the collar is a coil spring.

16. The method according to claim 12 wherein the collar is a shrink tubing and the step of positioning includes shrinking the shrink tubing about the proximal portion.

17. The method according to claim 12 wherein the step of removing includes machining the distal portion of the core wire with a multi-axis grinding machine to form the distal tip.

18. The method according to claim 12 further comprising removing material from a distal section of the proximal portion of the core wire to define a recessed portion configured for retaining the collar and the step of positioning includes placing the collar about the recessed portion, retaining the collar thereon.

19. The method according to claim 12 wherein the step of positioning includes attaching the collar to the proximal portion by one of adhesive bonding, soldering and welding.

20. The method according to claim 12 wherein the collar includes a radiopacifier that is detectable by at least one of X-ray and fluoroscopic visualization.

21. A guidewire comprising:
a core wire including a proximal portion and a distal tip extending therefrom, the distal tip exclusively formed from the core wire; and
a collar adjacent to the distal tip disposed about the proximal portion.

22. The guidewire according to claim 21 wherein the proximal portion has a recessed portion disposed adjacent to the distal tip, the recessed portion retaining the collar which is disposed thereabout.

23. The guidewire according to claim 21 wherein the distal tip has a maximum cross-sectional dimension not exceeding a maximum diameter of the proximal portion.

24. The guidewire according to claim 23 wherein the maximum diameter of the proximal portion does not exceeding about 0.020 inches.
